(19) United States
(12) Patent Application Publication
(10) Pub. No.: US 2019/0038750 A1
(43) Pub. Date: Feb. 7, 2019

(54) TRIPLE ANTIBIOTIC CATIONIC OINTMENT

(71) Applicant: Marc Selner, Studio City, CA (US)

(72) Inventor: Marc Selner, Studio City, CA (US)

(21) Appl. No.: 15/669,886

(22) Filed: Aug. 4, 2017

(51) Int. Cl. A61K 47/06; A61K 9/06; A61K 31/7036; A61K 38/12; A61K 31/155; A61K 31/14

(52) U.S. Cl. CPC A61K 47/06; A61K 9/06; A61K 31/14; A61K 38/12; A61K 31/155; A61K 31/7036

(57) ABSTRACT

The present disclosure generally relates to petrolatum-based compositions for suspension of active ingredients and methods for forming stable suspensions of active ingredients in petrolatum, and more specifically to compositions of triple antibiotic cationic ointments. In particular, the present disclosure relates to a method for suspension of antibiotics and biocides in petrolatum at room temperature and a petrolatum-based composition having a stable suspension of active ingredients including antibiotics and biocides.

### CROSS-REFERENCE TO RELATED APPLICATIONS

(Not applicable)

### STATEMENT REGARDING FEDERALLY SPONSORED RESEARCH OR DEVELOPMENT

(Not applicable)

### BACKGROUND OF THE INVENTION

### Field of the Invention

The present disclosure generally relates to petrolatum-based compositions for suspension of active ingredients and methods for forming stable suspensions of active ingredients in petrolatum, and more specifically to compositions of triple antibiotic cationic ointments. In particular, the present disclosure relates to a method for suspension of antibiotics and biocides in petrolatum at room temperature and a petrolatum-based composition having a stable suspension of active ingredients including antibiotics and biocides.

### Description of the Related Art

The statements in this section merely provide background information related to the present disclosure. This background discussion does not, however, establish or acknowledge such information as “prior art” to the disclosed compositions, systems and/or methods.

The preparation of emulsions and suspensions typically requires the use of one or more emulsifiers or other agents that serve to maintain the oil phase separated within the aqueous phase, or the aqueous phase separated within the oil phase. An emulsifier, as used herein, is an added formulation ingredient used to reduce the tension between hydrophilic and hydrophobic surface ingredients, thereby facilitating the mixture hydrophilic and hydrophobic ingredients.

A problem that is often encountered is the separation of the oil and aqueous phases over time, either by coalescing of the dispersed phase, or in some cases, by the combination of dispersed phase portions in a process of flocculation (formation of solid materials within the dispersion/suspension/emulsion that results in separation of the phases, with one in solid or semi-solid form.

Petrolatum is a semi-solid mixture of long chain (greater than C=20) hydrocarbons. Petrolatum is a known skin protectant and has been widely used in cosmetic and dermatological applications. Petrolatum is known to have advantageous protective properties when applied to the skin, however, its hydrophobic nature presents challenges with formulating active ingredients. Due to its hydrophobic nature, an emulsifier is generally used to disperse active ingredients throughout petrolatum. Petrolatum was also believed to be incapable of delivering active ingredients because the active ingredients would be trapped within the petrolatum and therefore would be unable to reach the desired delivery site.

Antimicrobial resistance (AMR) is the ability of a microbe to resist the effects of medication previously used to treat them, and includes antibiotic resistance, which applies to bacteria and antibiotics including methicillin-resistant Staphylococcus aureus (MRSA), vancomycin-resistant S. aureus (VRSA), extended spectrum beta-lactamase (ESBL), vancomycin-resistant Enterococcus (VRE), and multidrug-resistant A. baumannii (MRAB).

Combination antibiotic therapy is frequently used to treat severe Gram-negative infections but is controversial and debatable. Potential achievements with combinations as compared with monotherapy include a broader antibacterial spectrum, synergistic effects, and reduced risk for emerging resistance during therapy. In the absence of evidence-based treatment options, combinations are increasingly employed to enhance the antibacterial effects of available drugs against multidrug-resistant strains. However, excessive use of combinations should be avoided because it might be associated with increased risk for toxicity, superinfections, selection of resistant strains, and higher costs. Combination antibiotic therapy is recommended for severe sepsis and septic shock to reduce mortality related to inappropriate antibiotic treatment.

Antibiotics increase selective pressure in bacterial populations, causing vulnerable bacteria to die; this increases the percentage of resistant bacteria, which continue growing. With resistance to antibiotics becoming more common there is greater need for alternative treatments. Calls for new antibiotic therapies have been issued, but new drug development is becoming rarer.

The addition of biocides to a triple antibiotic ointment will increase coverage of bacteria and resistant organisms. Neomycin Sulfate and Polymixin B are cyclic peptides that kill micro-organisms with cations. Bacitracin zinc is derived from bacteria. Quaternary ammonias cationic biocides kill micro-organisms with cations—positively charged substances—and are both compatible within the triple antibiotic cationic ointment. Thus, there is a need for compositions of triple antibiotic cationic ointments and processes of making a stable suspension of active ingredients such as antibiotics and biocides. In addition, the petrolatum-based compositions described herein are gentle, do not irritate the skin, and are not cytotoxic to mammalian cells.

### BRIEF SUMMARY OF THE INVENTION

The present disclosure generally relates to petrolatum-based compositions for suspension of active ingredients and processes for forming stable suspensions of active ingredients in petrolatum, and more specifically to compositions of triple antibiotic cationic ointments. In particular, the present disclosure relates to a process for suspension of antibiotics and biocides in petrolatum at room temperature and a petrolatum-based composition having a stable suspension of active ingredients such as antibiotics and biocides.

Exemplary compositions and methods of triple antibiotic cationic ointments and methods of making the same are provided. According to an exemplary embodiment of the present disclosure, a composition for triple antibiotic cationic ointment is provided. The composition comprising: a petrolatum base; at least one antibiotic powder, the at least one antibiotic powder mixed into the petrolatum base to form an ointment; and at least one biocide, wherein the at least one biocide is added in nanodroplets to the ointment forming a stable suspension.

According to an exemplary embodiment of the present disclosure, a method for formulating a petrolatum-based composition is provided. The method comprises (a) mixing at least one antibiotic powder into a petrolatum base; and (b) adding nanodroplets of a liquid cationic biocide to the mixture, wherein the nanodroplets are suspended in a stable triple antibiotic cationic ointment.

According to an exemplary embodiment of the present disclosure, a composition for a biocide suspension ointment is provided. The composition comprises a petrolatum base; at least one powder-form ingredient, the at least one powder-form ingredient mixed into the petrolatum base to form an ointment; and at least one biocide, wherein the at least one biocide is added in nanodroplets to the ointment to form a stable suspension.

Exemplary embodiments of the present disclosure provide broader coverage and prevents resistance to antibiotics. Additional features, functions, and exemplary implementations of the disclosed compositions, systems, and methods will be apparent from the description which follows, particularly when read in conjunction with the appended drawings.

These and other objects, features, and attributes of the exemplary embodiments of the present disclosure, and its advantageous applications and/or uses, will become apparent from the following detailed description of the exemplary embodiments, when taken in conjunction with the appended drawings. It is to be understood, however, that the drawings are designed as an illustration only and not as a definition of the limits of the present disclosure.

### BRIEF DESCRIPTION OF THE DRAWINGS

Further objects, features and advantages of the present disclosure will become apparent from the following description taken in conjunction with the accompanying drawings, in which:

FIG. 1 depicts the chemical structure of Polyhexanide (polyhexamethylenebiguanide, PHMB) in accordance with an exemplary embodiments of the present disclosure;

FIG. 2 depicts the chemical structure of benzalkonium chloridein accordance with an exemplary embodiment of the present disclosure; and

FIG. 3 depicts the chemical structure of Polyaminopropylbiguanide (PAPB) in accordance with another exemplary embodiment of the present disclosure.

Throughout the drawings, the same reference numerals and characters, unless otherwise stated, are used to denote like features, elements, components, or portions of the illustrated embodiments and like reference numerals are used throughout the different views. Moreover, while the present disclosure will now be described in detail with reference to the figures, it is done so in connection with the illustrative embodiments and is not limited by the particular embodiments illustrated in the figures. It is intended that changes and modifications can be made to the described exemplary embodiments without departing from the true scope and spirit of the present disclosure as defined by the appended claims. The drawings are not necessarily to scale, emphasis instead being placed upon illustrating the principles of the invention.

### DETAILED DESCRIPTION OF THE EXEMPLARY EMBODIMENTS

Further areas of applicability of the present disclosure will become apparent from the description provided herein. It should be understood that the description includes specific examples that are intended for purposes of illustration and are not intended to limit the scope of the present teachings. As used herein, “at least one”, “a”, or “an” means one or more and includes individual components as well as mixtures or combinations of the individual components.

The present disclosure generally relates to petrolatum-based compositions for suspension of active ingredients and methods for formulating stable suspensions of active ingredients in petrolatum, and more specifically to compositions of triple antibiotic cationic ointments. In particular, the present disclosure relates to a method for suspension of antibiotics and biocides in petrolatum at room temperature and a petrolatum-based composition having a stable suspension of active ingredients including antibiotics and biocides.

Exemplary embodiments of the present disclosure provide a delivery system for active ingredients applicable to wide range of medical and cosmetic applications. The suspension of the present invention can be used to prepare ointments or other medicaments, useful in the treating of fungal, viral, and/or bacterial infections, and provide a time released biocide that does not wash away with exudate. The present invention biocidal suspensions permit the biocide to continue killing organisms over time without washing away or becoming depleted, and remaining in contact with the skin. The present invention suspension also allows drainage of the wound to occur to the outer dressing ideal for wound care.

The present disclosure provides for compositions that are petrolatum-based. Petrolatum, also known as petroleum jelly, white petrolatum, soft paraffin or multi-hydrocarbon, (CAS number 8009-03-8), is a semi-solid mixture of hydrocarbons (with carbon numbers mainly higher than 25), and is commercially available from a variety of sources.

According to exemplary embodiments, the petrolatum-based compositions described herein consist essentially of petrolatum, one or more active ingredients, and water. In other embodiments, the petrolatum-based compositions described herein consist of petrolatum, one or more active ingredient, and water. The active ingredient may be an antibiotic, a biologic, a vitamin or supplement, a pharmaceutical, or a cosmetic ingredient. In particular, the active ingredient may be a cationic biocide.

A petrolatum-based composition is made up primarily of petrolatum. The petrolatum is preferably medical grade petrolatum. According to exemplary embodiments, the petrolatum-based composition is greater than about 80% petrolatum.

According to an exemplary embodiment, the composition contains one or more antibiotics and one or more biocides. In particular, the petrolatum-based composition contains a triple antibiotic ointment.

In addition, the petrolatum-based composition also contains one or more biocide active ingredients dispersed throughout the triple antibiotic ointment. The biocide is cationic (positive charge) and attaches to lipid cell membranes of bacteria, yeast, fungus, and viruses.

Furthermore, the biocide is amphoteric which enables it to penetrate the biofilm that house bacteria. The biocide active ingredient does not react with the petrolatum. Instead, the biocide active ingredient is dispersed in the petrolatum as nanodroplets, and the petrolatum serves as a suspension matrix for the active ingredients. “Nanodroplet,” as used herein, is an aggregation of molecules of active ingredient in the petrolatum base. The nanodroplets typically contain a small amount of solvent in addition to the active ingredient. The nanodroplets may vary in size but generally the longest dimension of the nanodroplets measures from about 10 nm to about 10,000 nm.

The remaining weight of the composition, typically from about 0.1% to about 6% by weight of the petrolatum-based composition, is water or another solvent. In an exemplary embodiment, the composition contains about 5% water.

According to an exemplary embodiment of the present disclosure, a petrolatum-based composition comprising one or more antibiotics and a cationic biocide is provided. More specifically, the present disclosure provides for petrolatum-based compositions for the formulation of a triple antibiotic cationic ointment. The triple antibiotic cationic ointment comprises a triple antibiotic ointment and a liquid cationic biocide mixture dispersed therein.

The triple antibiotic ointment is a petrolatum base mixed with antibiotic powders. In particular, the triple antibiotic ointment may include the following three antibiotic powders: bacitracinzinc (“bacitracin”), neomycin sulfate (“neomycin”), and polymixin b. The antibiotic powders are added to petrolatum and optionally mineral oil. According to an exemplary embodiment, the general concentration of each ingredient per gram of petrolatum for the triple antibiotic ointment is as follows: bacitracin (˜400 units, up to 500 units), neomycin (3.5 mg/g), and polymixin b (˜5,000 units, up to 10,000 units).

Other antibiotic powders may be used including, but not limited to: tobramycin, erythromycin, Keflex, penicillin, doxycycline, minocin, Bactrim, and sulfonamide. Antibiotic powders may be sterilized for use ophthalmically.

Exemplary cationic biocides include, but are not limited to: Polyhexanide (polyhexamethylenebiguanide, PHMB), benzalkonium chloride, and Polyaminopropylbiguanide (PAPB). Other quaternary ammonium compounds may be used including, but not limited to, triclosan, betaines, and chlorhexidine. These cationic biocides have a positive charge that attach to the lipid cell membranes of bacteria, yeast, fungus, and viruses and are amphoteric which enables it to penetrate the biofilm that house bacteria. According to exemplary embodiments, the biocide is Polyhexanide (polyhexamethylenebiguanide, PHMB) or benzalkonium chloride, or a combination of PHMB and benzalkonium chloride.

FIG. 1 illustrates the structure of Polyhexanide (polyhexamethylenebiguanide, PHMB). Polyhexanide (polyhexamethylenebiguanide, PHMB) is a polymer used as a disinfectant and antiseptic. In dermatological use, it is spelled polihexanide (INN) and sold under names such as Lavasept, Serasept, and Omnicide. PHMB has been shown to be effective against Pseudomonas aeruginosa, Staphylococcus aureus (also the methicillin-resistant type, MRSA), Escherichia coli, Candida albicans (yeast), Aspergillus brasiliensis (mold), vancomycin-resistant enterococci, and Klebsiella pneumoniae (carbapenem-resistant enterobacteriaceae).

Some products containing PHMB are used for inter-operative irrigation, pre- and post-surgery skin and mucous membrane disinfection, post-operative dressings, surgical and non-surgical wound dressings, surgical bath/hydrotherapy, chronic wounds like diabetic foot ulcer and burn wound management, routine antisepsis during minor incisions, catheterization, scopy, first aid, surface disinfection, and linen disinfection. PHMB eye drops have been used as a treatment for eyes affected by Acanthamoeba keratitis. PHMB is also used as an ingredient in some contact lens cleaning products, cosmetics, personal deodorants and some veterinary products. The PHMB hydrochloride salt (solution) is used in the majority of formulations.

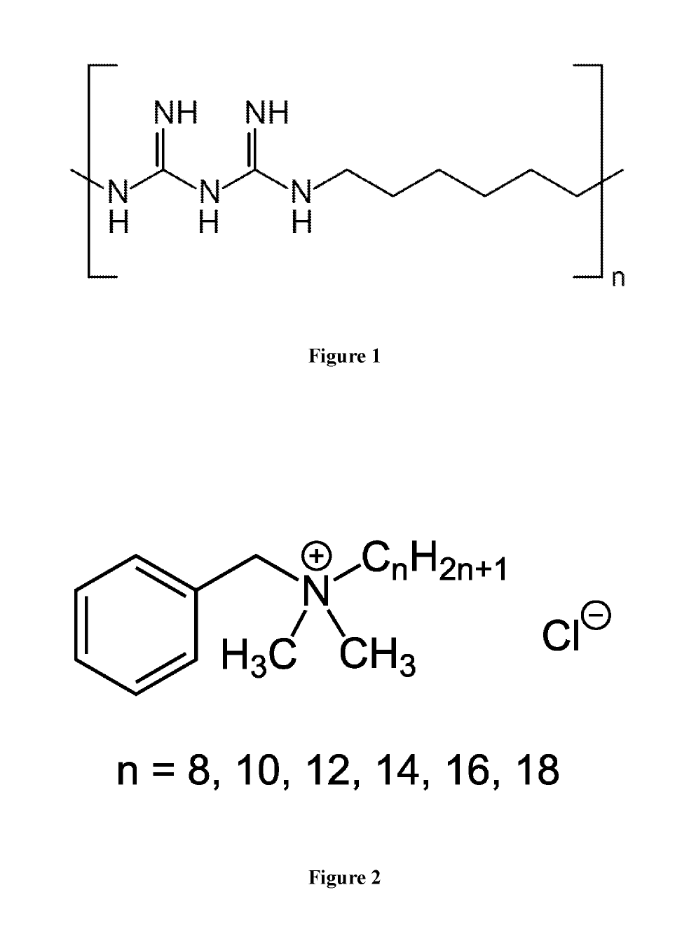

FIG. 2 illustrates the structure of benzalkonium chloride. Benzalkonium chloride is an organic salt known for its antimicrobial activity. Benzalkonium chloride is found in a variety of consumer products including skin antiseptics, disinfectant solutions (e.g. post-piercing disinfectant), over-the-counter single-application treatments for herpes, cold-sores, and fever blisters, throat lozenges and mouthwashes, and pharmaceutical products such as eye, ear and nasal drops or sprays. An advantage of benzalkonium chloride, not shared by ethanol-based antiseptics or hydrogen peroxide antiseptic, is that it does not cause a burning sensation when applied to broken skin.

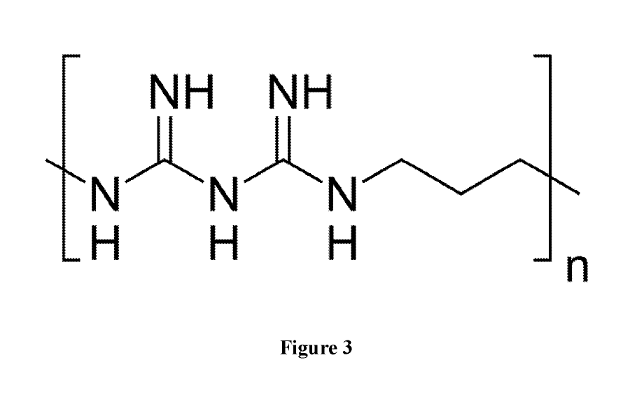

An alternative biocide is Polyaminopropylbiguanide (PAPB). FIG. 3 illustrates the structure of Polyaminopropylbiguanide (PAPB). PAPB is a disinfectant and a preservative used for disinfection on skin and in cleaning solutions for contact lenses. It is also an ingredient in many deodorant body sprays. It is a polymer or oligomer where biguanide functional groups are connected by hexyl hydrocarbon chains, with varying chain lengths. PAPB is specifically bactericidal at very low concentrations (10 mg/1) and is also fungicidal.

The biocide active ingredient may be a soluble ingredient. As used herein, a “soluble ingredient” refers to an ingredient (in its free base, free acid or salt form) having solubility in water or a polar solvent in excess of about 1 mg/ml at room temperature (20-25.degree.).

According to an exemplary embodiment, the cationic biocide may be a mixture of 0.025-5% PHMB with 0.13% benzalkonium chloride in water. In particular, the cationic biocide mixture may comprise 5% PHMB. However, experimentation demonstrates that the cationic biocide mixture may contain up to 20% PHMB and will combine with the triple antibiotic ointment at room temperature. The cationic biocide mixture is added, drop by drop, to the triple antibiotic ointment to form a 1-5% addition of the biocide per gram of triple antibiotic ointment.

The active ingredients of the present disclosure are dispersed throughout the petrolatum as nanodroplets which mixes with the powder(s). The petrolatum and powders serves as a suspension matrix for the active ingredients. Notably, petrolatum-based compositions of the present disclosure do not contain an emulsifier. The active ingredients are dispersed in nanodroplets into petrolatum-based triple antibiotic ointment and result in a stable suspension. The compositions of the present disclosure are shelf stable for extended periods of time, as well as in unideal conditions. Importantly, the compositions exhibit exceptional chemical stability of active ingredients and enable deliver of active ingredients over extended periods of time.

The compositions, more specifically the triple antibiotic cationic ointments, described herein are stable. In one aspect, stability refers to the integrity of the composition as a whole, and in particular, the stability of the nanodroplets in the petrolatum. Under ambient conditions, the petrolatum and the active ingredients will not separate for greater than two years meaning that the composition is shelf stable for at least two years. Even under accelerated conditions, such as reduced pressure, the petrolatum and the active ingredients do not separate. In addition to the stability of the composition, the compositions also show exceptional chemical stability for the active ingredient. The chemical stability stems primarily from the low-temperature manufacturing process described below. The absence of excessive heat conditions in the manufacturing of the compositions improves the chemical stability (resistance to degradation) for the active ingredients.

According to exemplary embodiments, the petrolatum-based compositions described herein consist of petrolatum, one or more active ingredients including a powder-form active ingredient and a liquid biocide active ingredient, and water. Although reference is made throughout the disclosure to exemplary embodiments comprising a triple antibiotic ointment (three antibiotic powders) and a liquid biocide mixture (added in nanodroplets and suspended in the ointment), it should be understood that the composition may comprise at least one powder-form active ingredient (i.e., a powder-form active ingredient, not only antibiotic powders). For example, potential powder-form active ingredients include, but are not limited to: Neomycin Sulfate, Bacitracin zinc, Polymixin b, cortisone, Cyclosporin. Next, a water solution of one or more cationic biocides such as PHMB, bezalkonium chloride in a concentration of 0.0025% to 5%, without heating the liquid or heating the petrolatum to the melting point.

Using the same process of adding one or more powders of baking soda, collagenase in petrolatum to a water anionic solution of sodium hypochlorite in a concentration of 0.0025% to 5%, without heating the liquid or petrolatum to the melting point.

Experiments shows that adding a biocide(s) to a water soluble solution liquid to a room temperature mixture of petrolatum and triple antibiotic mixture, or single antibiotic mixing, or cortisone or cyclosporine mixture in petrolatum will permanently mix and not separate. The reason for this is that the powder mixture combines with the liquid like an emulsifier, but is not an emulsion, which typically has at one end of the molecule water-soluble end and the other a fat-soluble end, and as a result, permanently suspends the two without the need for heat. You can call this absorbs, mixes, forms together. The mixture can then be heated below the melting point to improve flow and resistance for filling purposes. The powder(s) first must be added to the petrolatum and mixed. To the resulting mixture a water soluble mixture of biocide/s and water can be added and permanently mixed. Normally if a liquid were added to petrolatum at room temperature, the liquid would separate or coalesce. By adding the powder to the petrolatum first and mixing, the powder is then able to combine subsequently with the water solution and biocide(s). Experimentation and study proved the theory dozens of times with different powders and even month later the mixture stays mixed without any separation of liquid even when the tubes are placed vertical for long periods of time. This is a test for separation, since if there were separation, the liquid being heavier than the petrolatum would go to the bottom of the tube and upon opening the tube the liquid would be visibly separated. Of course, emulsifiers can hold oil and water together. However, this is not an emulsifier, but to the degree it is able to combine or absorb or mix, it acts like an emulsifier.

The compositions may be applied the desired site for delivery of the active ingredients. Generally, the application is topical to the skin, but the compositions may also be used intraoperatively. Additionally, the compositions may be incorporated in predetermined therapeutically effective amounts into disposables such as wipes, gauze, patches, wraps, bandages, adhesive strips, sponge, cotton swab, sutures, medication pad, etc.

The present disclosure also provides for a method for formulating the petrolatum-based compositions. More specifically, the method provides for the formulation of petrolatum-based compositions comprising one or more antibiotics and a cationic biocide.

According to an exemplary embodiment of the present disclosure, the method comprises: (a) mixing one or more antibiotic active ingredients in powder-form to petrolatum to make a triple antibiotic ointment; and (b) adding, drop by drop, a liquid cationic biocide mixture to the triple antibiotic ointment to make a triple antibiotic cationic ointment. Steps (a)-(b) are conducted in sequence. Each step is conducted at room temperature, without heating the ingredients. According to exemplary embodiments, one drop of liquid biocide mixture adds a 5% addition of biocide per gram of triple antibiotic ointment.

The method involves the use of nanodroplets of the liquid biocide mixture, which combine with the powder to form a water-soluble suspension. In particular, similar to an emulsion, the liquid biocide mixture absorbs and is held by the powder. The antibiotic powders, that are mixed into the petrolatum-base, attracts the nanodroplets of the liquid biocide mixture and the nanodroplets combine with/dissolve into the powder. As a result, the liquid biocide mixture is suspended in the triple antibiotic cationic ointment.

Other methods may be used to formulate a petrolatum-based composition having a stable suspension of active ingredients including antibiotics and biocides by adding Neosporin to omnibiocide gel. However, the concentration of the triple antibiotic ointment would have to be doubled for the preferred concentration of antibiotics. An alternative method includes adding the triple antibiotic powder to a mixture of omnibiocide. Of course then the mixture would be a powder in liquid, whereas the patent describes a process of using a liquid biocides directly suspended in petrolatum without the use of any emulsifier or emulsifier like formulation.

While illustrative embodiments of the invention have been described, it is noted that various modifications will be apparent to those of ordinary skill in the art in view of the above description and drawings. Such modifications are within the scope of the invention, which is limited and defined only by the following claims.

1. A composition for a triple antibiotic cationic ointment comprising:
a petrolatum base;
at least one antibiotic powder, the at least one antibiotic powder mixed into the petrolatum base to form an ointment;
at least one biocide solution, wherein the at least one biocide is added in nanodroplets to the ointment forming a stable suspension; and
water.

2. The petrolatum-based composition of claim 1, wherein the at least one antibiotic powder includes neomycin sulfate, bacitracin zinc, and polymixin b.

3. The petrolatum-based composition of claim 2, wherein the concentration of each of the antibiotic powders per gram of petrolatum includes 3.5 mg of neomycin, 400 units of bacitracin zinc, and 5,000 units of polymixin b.

4. The petrolatum-based composition of claim 1, wherein the at least one biocide is a cationic quaternary ammonium.

5. The petrolatum-based composition of claim 1, wherein the at least one biocide is PHMB.

6. The petrolatum-based composition of claim 1, wherein the at least one biocide is benzalkonium chloride.

7. The petrolatum-based composition of claim 1, wherein the at least one biocide includes both PHMB and benzalkonium chloride.

8. A composition for a biocide suspension ointment comprising:
a petrolatum base;
at least one powder-form ingredient, the at least one powder-form ingredient mixed into the petrolatum base to form an ointment; and
at least one biocide in water soluble liquid, wherein the at least one biocide is added in nanodroplets to the ointment to form a stable suspension.

9. The composition for a biocide suspension ointment of claim 8, wherein the at least one powder-form ingredient is from the group consisting of neomycin sulfate, polymyxin b, bacitracin, cortisone, cyclosporine, and baking powder.

10. A method for formulating a petrolatum-based composition comprising:
(a) mixing at least one powder-form ingredient into a petrolatum base; and
(b) adding nanodroplets of a water soluble liquid biocide solution to the mixture, wherein the nanodroplets are suspended in a stable triple antibiotic cationic ointment.

11. The method for formulating a petrolatum-based composition of claim 10, wherein the powder-form ingredient is one of an antibiotic powder, a cortisone powder, a cyclosporine powder, or a baking soda powder.

12. The method for formulating a petrolatum-based composition of claim 10, wherein the liquid biocide solution consists of cationic biocides.

13. The method for formulating a petrolatum-based composition of claim 10, wherein the powder-form ingredient is the baking soda powder and the liquid biocide solution is an anionic sodium hypochlorite.
